(19) United States
(12) Patent Application Publication
(10) Pub. No.: US 2018/0369272 A1
(43) Pub. Date: Dec. 27, 2018

(54) Biochemical Scaffolds for Modulating Cell Function

(71) Applicant: Next2Me, LLC, Oakland, CA (US)

(72) Inventors: Ralph L. Peterson, Oakland, CA (US); Renee D. Williman, Oakland, CA (US)

(21) Appl. No.: 16/116,539

(22) Filed: Aug. 29, 2018

Related U.S. Application Data
(63) Continuation-in-part of application No. 14/223,392, filed on Mar. 24, 2014.

(51) Int. Cl. A61K 31/714; A61K 36/81; A61K 31/4375; A61K 36/296; A61K 31/05; A61K 36/79; A61K 36/254; A61K 36/31; A61K 31/198

(52) U.S. Cl. CPC A61K 31/714; A61K 36/81; A61K 31/4375; A61K 36/296; A61K 31/198; A61K 36/79; A61K 36/254; A61K 36/31; A61K 31/05

(57) ABSTRACT

A biochemical scaffold for regulating mammalian cell function, including a bioenergetic platform and a vibrational platform, the bioenergetic platform comprising a Krebs cycle modulator and/or glutathione modulator and/or neurotransmitter modulator and/or DNA modulator and/or endocannabinoid system modulator, the vibrational platform comprising at least one energy signature component, e.g. herb, that is subjected to harmonic oscillation in the range of approximately 23 Hz-1000 GHz for a period of time in the range of approximately 3-48 hrs.

### CROSS-REFERENCES TO RELATED APPLICATIONS

This application is a continuation-in-part of U.S. application Ser. No. 14/223,392, filed on Mar. 24, 2014, which claims the benefit of U.S. Provisional Application No. 61/936,116, filed on Feb. 5, 2014.

### FIELD OF THE INVENTION

The present invention relates to compositions and methods for inducing cell activity. More particularly, the present invention relates to biochemical scaffolds and associated methods for inducing, supporting and/or enhancing cell activity and, thereby, function.

### BACKGROUND OF THE INVENTION

As is well known in the art, optimal cell activity and, hence, function is essential to human existence. Cell activity and function is primarily dependent on the energy potential of a cell. Where cellular energy has been reduced, a cascade of undesirable cellular events can, and often times will, result. The noted cellular events typically result in one or more undesirable physiological characteristics, such as reduced stamina or endurance, and mental clarity.

Reduction of cellular energy can also result in dysfunction of various organs, e.g., heart and/or liver failure. When cellular energy approaches zero, cell death, i.e. apoptosis, is often encountered.

As is also well known in the art, cellular energy is directly dependent on various biochemical processes; particularly, cell respiration, i.e. metabolic reactions and processes that take place in the cells to convert biochemical energy from nutrients into adenosine triphosphate (ATP).

The metabolic reactions and processes, which are often referred to as a metabolic pathway, are typically embodied in the Krebs cycle.

Referring to FIG. 1, there is shown a schematic illustration of a Krebs cycle. As illustrated in FIG. 1, through catabolism of carbohydrates, fats and proteins, a two carbon organic product, i.e. acetate in the form of acetyl-CoA, is produced. Acetyl-CoA and two equivalents of water (H₂O) are consumed during the citric acid cycle, producing two equivalents of carbon dioxide (CO₂) and one equivalent of HS-CoA.

In addition, one complete evolution of the Kreb cycle converts three equivalents of nicotinamide adenine dinucleotide (NAD⁺) into three equivalents of reduced NAD⁺ (NADH), one equivalent of ubiquinone (Q) into one equivalent of reduced ubiquinone (QH₂), and one equivalent each of guanosine diphosphate (GDP) and inorganic phosphate (Pᵢ) into one equivalent of guanosine triphosphate (GTP). The NADH and QH₂ generated during the Kreb cycle are in turn used by the oxidative phosphorylation pathway to generate energy-rich adenosine triphosphate (ATP).

A primary source of acetyl-CoA is carbohydrates, which are broken down by glycolysis to produce pyruvate. Pyruvate, in turn, is decarboxylated by the enzyme pyruvate dehydrogenase. The decarboxylated pyruvate generates acetyl-CoA, according to the following equation:

CH₃C(═O)C(═O)O⁻+HSCoA+NAD⁺→CH₃C(═O)SCoA+NADH+H⁺+CO₂
where
CH₃C(═O)C(═O)O⁻ represents pyruvate; and
CH₃C(═O)SCoA represents acetyl-CoA.

Regulation of the Krebs cycle is largely dependent upon product inhibition and substrate availability. For example, NADH, a product of all dehydrogenases in the cycle (with the exception of succinate dehydrogenase) inhibits pyruvate dehydrogenase, isocitrate dehydrogenase, α-ketoglutarate dehydrogenase, and citrate synthase. Acetyl-coA inhibits pyruvate dehydrogenase, while succinyl-CoA inhibits alpha-ketoglutarate dehydrogenase and citrate synthase.

Various elements and compositions have thus been employed to modulate one or more Krebs cycle processes to enhance cell activity and, thereby, generation of ATP. For example, calcium has been successfully employed to regulate the Krebs cycle. Calcium activates pyruvate dehydrogenase phosphatase, which, in turn, activates the pyruvate dehydrogenase complex. Calcium also activates isocitrate dehydrogenase and α-ketoglutarate dehydrogenase. This increases the reaction rate of many of the sequences in the cycle, and therefore increases flux throughout the pathway.

Citrate has also been employed as a feedback inhibitor. Citrate inhibits phosphofructokinase, i.e. an enzyme involved in glycolysis that catalyses formation of fructose 1,6-bisphosphate, which is a precursor of pyruvate. This inhibits the formation of a high rate of flux when there is an accumulation of citrate.

Recent efforts have also been directed to the link between intermediates of the Krebs cycle and the regulation of hypoxia-inducible factors (HIF). HIF plays a role in the regulation of oxygen homeostasis, and is a transcription factor that targets angiogenesis, vascular remodeling, glucose utilization, iron transport and apoptosis.

HIF is synthesized consititutively. Hydroxylation of at least one of two critical proline residues also mediates their interaction with the von Hippel Lindau E3 ubiquitin ligase complex, which targets them for rapid degradation. This reaction is catalyzed by prolyl 4-hydroxylases.

Various elements and compositions, such as fumarate and succinate, have thus been employed in an effort to inhibit the formation of prolyl hydroxylases and, thereby, stabilize HIF.

Although some of the noted elements and compositions have garnered some success in inducing Krebs cycle activity and, thereby cell activity and function (and, hence, enhancing ATP energy), there remains a need for improved biochemical formulations that effectively and readily enhance cell activity by inducing and/or modulating multiple Krebs cycle reactions and/or pathways.

Various formulations and efforts have also been employed to enhance cell activity and function by inducing or modulating other molecular actions, including inducing the generation and transmission of electrochemical signals, i.e. neurotransmitters, inducing DNA activity and inducing and/or modulating cell receptor activity.

Although the noted efforts have similarly garnered some success in enhancing cell activity and function, there still remains a need for biochemical scaffolds that effectively and readily enhance cell activity and, thereby optimal cell function, by inducing and modulating a plurality of seminal cell activities.

It would thus be desirable to provide improved biochemical scaffolds, i.e. formulations, and methods that enhance cell activity and function and, thereby, physical and mental function, by modulating multiple Krebs cycle reactions and/or pathways.

It would also be desirable to provide biochemical scaffolds and methods that enhance cell activity and function and, thereby, physical and mental function, by inducing (i) the generation of electrochemical signals, i.e. neurotransmitters, and modulating the transmission thereof by and between neurons, (ii) DNA activity and (iii) cell receptor activity.

It would also be desirable to provide biochemical scaffolds that enhance cell activity and function and, thereby, physical and mental function, by inducing the generation and proliferation of selective cells and associated elements.

It is therefore an object of the present invention to provide biochemical scaffolds that enhance cell activity and function and, thereby, physical and mental function, by modulating at least one Krebs cycle metabolic reaction, process and/or pathway.

It is another object of the present invention to provide biochemical scaffolds that enhance cell activity and function and, thereby, physical and mental function, by inducing the generation of neurotransmitters and modulating the transmission thereof by and between neurons.

It is another object of the present invention to provide biochemical scaffolds that enhance cell activity and function and, thereby, physical and mental function, by inducing cell receptor activity.

It is another object of the present invention to provide biochemical scaffolds that enhance cell activity and function and, thereby, physical and mental function, by inducing and/or modulating cell receptor activity.

It is another object of the present invention to provide biochemical scaffolds that enhance cell activity and function and, thereby, physical and mental function, by inducing and/or modulating endocannabinoid system activity.

It is another object of the present invention to provide biochemical scaffolds that support and/or enhance mitochondrial DNA activity.

It is another object of the present invention to provide biochemical scaffolds that enhance cell activity and function and, thereby, physical and mental function, by modulating a plurality of cell activities.

### SUMMARY OF THE INVENTION

The present invention is directed to biochemical scaffolds and associated methods that induce and/or modulate at least one, more preferably, a plurality of molecular activities, including, without limitation, inducing (i) at least one Krebs cycle metabolic reaction, process and/or pathway, (ii) generation or proliferation of glutathione and/or a member of the glutathione family, (ii) generation or proliferation of at least one neurotransmitter, and/or modulating the transmission thereof by and between neurons, (iv) inducing and/or supporting mitochondrial DNA activity and (v) cell receptor activity.

By virtue of the noted modulated molecular activities that are induced by the biochemical scaffolds of the invention, cellular function and, thereby, physical and mental function, is significantly enhanced. It has also been found that administration of the biochemical scaffolds of the invention to a subject can, and in many instances will ameliorate various physical disorders, including neuropathic pain, inflammation and core temperature spikes, and mental disorders, such as post-traumatic stress disorder (PTSD), depression and anxiety. The biochemical scaffolds of the invention can also be employed to abate drug dependence; particularly, opioid and heroin dependence.

In some embodiments of the invention, the biochemical scaffolds comprise two platforms: a vibrational energy platform and a bioenergetic platform.

In a preferred embodiment of the invention, the vibrational energy platforms comprise at least one laser activated biologically targeted energy blank or signature.

In some embodiments of the invention, the bioenergetic platforms comprise a specially formulated complex proprietary liquid herbal blend, i.e. a tincture, of oxygen enriched glycerin infused water molecules, and a specific assortment of complex-B vitamins.

In a preferred embodiment of the invention, the bioenergetic platforms comprise at least one Krebs cycle modulator, glutathione modulator, neurotransmitter modulator, DNA modulator or endocannabinoid modulator.

In some embodiments of the invention, the bioenergetic platforms comprise a mixture comprising two or more of the noted modulators.

In one preferred embodiment of the invention, the bioenergetic platforms comprise at least one Krebs cycle modulator, neurotransmitter modulator, and endocannabinoid modulator.

As discussed herein, it has been found that a unique synergism by and between selective Krebs cycle, neurotransmitter and endocannabinoid modulators exists, which unexpectedly induces a significantly enhanced level of cell activity and, hence, function.

In a preferred embodiment of the invention, the Kreb cycle modulators induce and/or modulate at least one Krebs cycle metabolic reaction, process and/or pathway, including, without limitation, Krebs cycle product inhibition and/or substrate availability.

In some embodiments, the Kreb cycle modulators also induce the production of CO₂, acetyl-CoA, FADH₂ and/or adenosine triphosphate (ATP).

In some embodiments of the invention, the Krebs cycle modulators comprise, without limitation, ashwaganda, eleuthero root (or extract), maca, an amino acid, e.g., L-arginine and L-citrulline, and vitamins B₂, B₁, B₃, B₅ and B₉.

In a preferred embodiment, the glutathione modulators induce the generation or proliferation of glutathione and/or a member of the glutathione family, including, without limitation, glutathione peroxidase, and/or catalase synthesis.

In some embodiments of the invention, the glutathione modulators comprise, without limitation, schisandra chinensis berry, damiana and epimedium, maca, nettle leaf, Fe and Cu, and B-vitamins selected from the group comprising B₂, B₅, B₆ and B₇.

In a preferred embodiment of the invention, the neurotransmitter modulators induce the generation of electrochemical signals, i.e. neurotransmitters, and/or modulate the transmission thereof by and between neurons and, hence, cells.

In some embodiments of the invention, the neurotransmitter modulators comprise, without limitation, nettle leaf, maca, eleuthero root, Yohimbe, cannabidiol (CBD), epimedium, and vitamins B₁ and B₆.

In a preferred embodiment of the invention, the DNA modulators support and/or enhance mitochondrial DNA activity.

In some embodiments, the DNA modulators support and/or enhance mitochondrial DNA activity by protecting and/or facilitating the repair of mitochondrial DNA.

In some embodiments of the invention, the DNA modulators comprise, without limitation, vitamin B₁₂.

In a preferred embodiment of the invention, the endocannabinoid system modulators induce cell receptor activity.

In a preferred embodiment, the endocannabinoid system modulators induce cannabinoid receptor activity.

In a preferred embodiment, the endocannabinoid system modulators comprise cannabidiol (CBD).

In some embodiments, the bioenergetic platform includes a cofactor, including, without limitation, organic cofactors, such as flavin and heme, and inorganic cofactor, such as metal ions of magnesium (Mg²⁺), copper (Cu⁺), manganese (Mn²⁺), and iron-sulfur clusters.

In a preferred embodiment of the invention, the vibrational energy platform comprises energy signature components (or extracts) derived from selective herbs and biological agents, including, without limitation, schisandra chinensis, damiana leaf, eleuthero root, stinging nettle leaf, maca root, yohimbe root, epimedium, L-arginine, and L-citrulline.

In some embodiments, the biochemical platforms further comprise a pharmacological agent or composition that induces or modulates a physiological or biological process, or cellular activity, e.g., induces proliferation, and/or growth and/or regeneration of cells.

According to the invention, the biochemical platforms can be delivered to host tissue by various conventional means, including, without limitation, oral, sublingual, nasal, direct injection, topical application, etc.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a Krebs cycle;

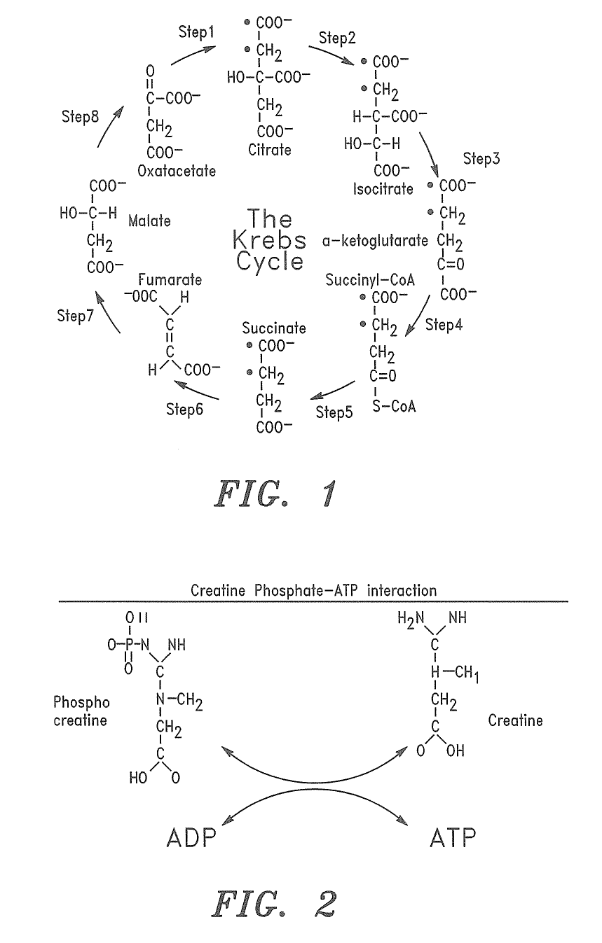

FIG. 2 is a schematic illustration of creatine phosphate-ATP interaction;

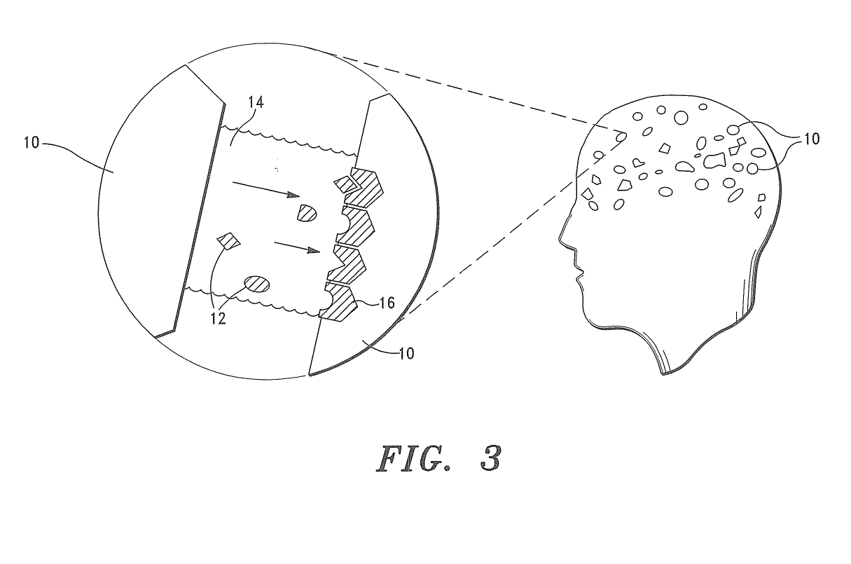

FIG. 3 is a schematic illustration of electrochemical signal transmission; and

FIGS. 4A and 4B are tables of biochemical scaffolds, according to the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The term “vibrational energy platform,” as used herein, means and includes biologically targeted complex, stable, and efficient energetic blanks and glycerol water-soluble molecules, which, when programmed with a laser charged imprint of herbs, minerals, vitamins, amino acids, or pharmaceutical properties (creating energy-signature templates), help stimulate/enable/enhance vital cellular biochemical processes necessary to maintain homeostasis.

The term “Krebs cycle modulator,” as used herein, means and includes an element, agent, drug, compound, composition of matter or mixture thereof, including its formulation, which induces and/or modulates a Krebs cycle metabolic reaction, process and/or pathway, including, without limitation, Krebs cycle product inhibition and/or substrate availability. According to the invention, suitable Krebs cycle modulators comprise, without limitation, eleuthero root (or extract), maca, an amino acid, e.g., L-arginine and L-citrulline, and vitamins B₂, B₁, B₃, B₅ and B₉ vitamins B2, B1, B3, B5 and B9.

The term “neurotransmitter modulator,” as used herein, means and includes an element, agent, drug, compound, composition of matter or mixture thereof, including its formulation, which induces the generation or proliferation of at least one neurotransmitter and/or modulates the transmission thereof by and between neurons and, hence, cells. According to the invention, suitable neurotransmitter modulators comprise, without limitation, nettle leaf, maca, eleuthero root, Yohimbe, and vitamins B₁ and B₆.

The term “glutathione modulator,” as used herein, means and includes an element, agent, drug, compound, composition of matter or mixture thereof, including its formulation, which induces the generation or proliferation of glutathione and/or the glutathione family, including, without limitation, glutathione peroxidase.

The term “glutathione modulator” also means and includes an element, agent, drug, compound, composition of matter or mixture thereof, including its formulation, which induces catalase synthesis.

According to the invention, suitable glutathione modulators comprise, without limitation, schisandra chinensis berry, damiana and epimedium, maca, nettles leaves, iron (Fe) and copper (Cu), and B-vitamins selected from the group comprising B₂, B₅, B₆ and B₇.

The term “DNA modulator,” as used herein, means and includes an element, agent, drug, compound, composition of matter or mixture thereof, including its formulation, that induces and/or modulates mitochondrial DNA, including protecting and/or facilitating the repair of mitochondrial DNA. According to the invention, a suitable DNA modulator comprises, without limitation, vitamin B₁₂.

The term “endocannabinoid system modulator,” as used herein, means and includes an element, agent, drug, compound, composition of matter or mixture thereof, including its formulation, which induces and/or modulates cell receptor activity; particularly, a cannabinoid receptor, i.e. CB1 or CB2. According to the invention, a suitable endocannabinoid system modulator comprises, without limitation, cannabidiol (CBD).

The terms “cellular dysfunction” and “cell dysfunction” are used interchangeably herein and mean and include a reduction or impairment in physical structure or function of a cell.

The term “organ dysfunction”, as used herein, means and includes a reduction or impairment in physical structure or function of a mammalian organ, including, without limitation, the cardiovascular vascular system (heart and lungs), digestive system (salivary glands, esophagus, stomach, liver, gallbladder, pancreas, intestines, colon, rectum and anus), endocrine system (hypothalamus, pituitary gland, pineal body, thyroid, parathyroids and adrenals), excretory system (kidneys, ureters, bladder and urethra), immune system (lymphatic system, tonsils, adenoids, thymus and spleen), integumentary system (skin, hair and nails), muscular system, nervous system (brain and spinal cord), reproductive system (ovaries, fallopian tubes, uterus, vagina, mammary glands, prostate and penis), respiratory system (pharynx, larynx, trachea, bronchi and diaphragm) and the skeletal system (bones, cartilage, ligaments and tendons).

The terms “prevent” and “preventing” are used interchangeably herein, and mean and include reducing the frequency or severity of a disease, condition, dysfunction or disorder. The term does not require an absolute preclusion of the disease, condition, dysfunction or disorder. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, dysfunction or disorder. The terms include “active treatment”, i.e. treatment directed specifically toward the improvement of a disease, pathological condition, dysfunction or disorder, and “causal treatment”, i.e. treatment directed toward removal of the cause of the associated disease, pathological condition, dysfunction or disorder.

The terms “treat” and “treatment” further include “palliative treatment”, i.e. treatment designed for the relief of symptoms rather than the curing of the disease, pathological condition, dysfunction or disorder, “preventative treatment”, i.e. treatment directed to minimizing or partially or completely inhibiting the development of the associated disease, pathological condition, dysfunction or disorder, and “supportive treatment”, i.e. treatment employed to supplement another specific therapy directed toward the improvement of the associated disease, pathological condition, dysfunction or disorder.

The terms “pharmacological agent,” “active agent” and “drug” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent,” “active agent” and “drug” thus mean and include, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombotic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, and vasodilating agents.

The term “therapeutically effective”, as used herein, means that the amount of a Krebs cycle modulator, glutathione modulator, neurotransmitter modulator or DNA modulator and/or biochemical scaffold formed therefrom or pharmacological or bioactive agent administered to a subject is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “delivery” and “administration” are used interchangeably herein, and mean and include providing a Krebs cycle modulator, glutathione modulator, neurotransmitter modulator or DNA modulator and/or biochemical scaffold formed therefrom to a subject through any method appropriate to deliver formulations and/or scaffolds to a subject. Non-limiting examples of delivery methods include oral, sublingual, nasal, direct injection, topical application, etc.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As indicated above, the present invention is directed to biochemical scaffolds and associated methods that induce and/or modulate at least one, more preferably, a plurality of molecular activities, including, without limitation, inducing (i) at least one Krebs cycle metabolic reaction, process and/or pathway, (ii) generation or proliferation of glutathione and/or a member of the glutathione family, (iii) generation or proliferation of at least one neurotransmitter, and/or modulating the transmission thereof by and between neurons, (iv) inducing and/or supporting mitochondrial DNA activity and (v) cell receptor activity.

As also indicated above, by virtue of the noted modulated molecular activities that are induced by the biochemical scaffolds of the invention, cellular activity and function and, thereby, physical and mental function, is significantly enhanced. It has also been found that administration of the biochemical scaffolds of the invention to a subject can, and in many instances will ameliorate various physical disorders, including neuropathic pain, inflammation and core temperature spikes, and mental disorders, such as post-traumatic stress disorder (PTSD), depression and anxiety. The biochemical scaffolds of the invention can also be employed to abate drug dependence; particularly, opioid and heroin dependence.

In a preferred embodiment of the invention, the biochemical scaffolds comprise two platforms (and components associated therewith): a vibrational energy platform and a bioenergetic platform.

In some embodiments of the invention, the bioenergetic platforms comprise a composition comprising oxygen enriched glycerin infused water molecules.

In some embodiments, the bioenergetic platforms comprise an herbal/vitamin composition comprising selective herbs, e.g., eleuthero root and Yohimbe, and B-vitamins.

As indicated above, in a preferred embodiment of the invention, the bioenergetic platforms comprise at least one Krebs cycle modulator, glutathione modulator, neurotransmitter modulator, DNA modulator or endocannabinoid modulator.

Thus, in some embodiments, the bioenergetic platforms comprise a Krebs cycle modulator and/or glutathione modulator and/or neurotransmitter modulator and/or DNA modulator and/or endocannabinoid modulator.

In some embodiments, the bioenergetic platforms comprise a plurality of Krebs cycle modulators and/or glutathione modulators and/or neurotransmitter modulators and/or DNA modulators and/or endocannabinoid modulators.

In a preferred embodiment of the invention, the Kreb cycle modulators induce and/or modulate a Krebs cycle metabolic reaction, process and/or pathway, including, without limitation, Krebs cycle product inhibition and/or substrate availability.

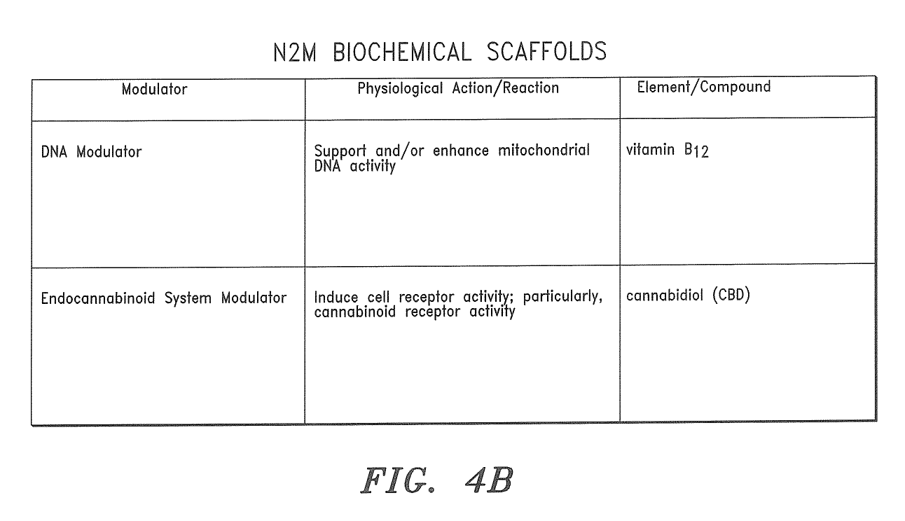

As set forth in FIG. 4 and discussed in detail below, in some embodiments, the Kreb cycle modulators also induce multiple Krebs cycle reactions and/or pathways, resulting in the production of CO₂, and/or acetyl-CoA, and/or FADH₂, and enhanced adenosine-5′-triphosphate (ATP) energy potential.

As set forth in Applicant's Co-pending U.S. application Ser. No. 14/223,392, ATP is a multifunctional nucleoside triphosphate that is used as a coenzyme in cells. ATP is one of the end products of photophosphorylation and cellular respiration, and is used by structural proteins in many cellular processes, including biosynthetic reactions, motility, and cell division.

Mammalian mitochondria are organelles that produce more than 90% of cellular ATP. In addition to supplying ATP, i.e. cellular energy, mitochondria are also involved in other cellular mechanisms, including cellular differentiation, apoptosis, as well as cell cycle modulation and cell growth.

Mitochondria provide intracellular ATP via a process called glycolysis, which breaks down monosaccharides into ATP through a series of biochemical processes. Mitochondria contain, among other things, the Krebs cycle enzymes that are involved in heme biosynthesis and the electron transport chain, i.e. the Oxidative Phosphorylation pathway (OxPHOS) system. Due to the large flux of redox reactions necessary to maintain oxidative phosphorylation, mitochondria are the primary site of production of reactive oxygen species (ROS).

It has, however, been found that increased production of ROS and interference with the OxPhos system can cause cell cycle dysfunction and arrest.

The OxPHOS system is composed of five large multi-protein enzyme complexes, which collectively transform the reducing energy of NADH and FADH₂ to ATP. NADH ubiquinone oxidoreductase (Complex I) contains 45 different subunits, and succinate ubiquinone reductase (Complex II), ubiquinone-cytochrome c oxidoreductase (Complex III), cytochrome c oxidase (Complex IV) and the ATP synthase (Complex V) have 4, 11, 13 and 16 subunits, respectively.

Four of the OxPHOS enzyme complexes (Complexes I, III, IV and V) have a dual genetic origin, i.e. they are composed of both nuclear DNA-encoded proteins and mitochondrial DNA-encoded proteins.

Transient ischemia (anoxia) results in the local production of extremely high levels of ROS, which can cause long term damage to mitochondria. In the initial phase of transient ischemia, oxygen is scarce, but tissue demands for ATP remain high, resulting in continued functioning of the OxPhos system except for the terminal reduction of oxygen to water by Complex IV. Therefore, reduced electron acceptors “upstream” of Complex IV accumulate to abnormally high levels.

Upon resupply of oxygen, these excess reduced carriers react directly with oxygen to generate highly toxic partially reduced oxygen species, which are capable of protein, lipid and DNA modifying reactions. The resulting oxidative damage is deemed to occur mainly inside the mitochondrion, because such radicals are so reactive that they are short lived and cannot diffuse far before finding a target for reaction.

Accordingly, OxPHOS proteins and intDNA are deemed the cellular molecules most affected by such oxidative stress. The resulting defects in intDNA and OxPHOS proteins can, and in most instances will, result in continued increased production of ROS.

However, it has been found that modulating the OxPhos system and, thereby, ROS production, which can be achieved by the Krebs cycle modulators of the invention, oxidative stress of cells can be substantially reduced or eliminated.

As discussed in detail herein. the Krebs cycle modulators of the invention preferably comprise, without limitation, ashwaganda, eleuthero root (or extract), maca, an amino acid, e.g., L-arginine and L-citrulline, and vitamins B₂, B₁, B₃, B₅ and B₉.

In a preferred embodiment of the invention, the glutathione modulators of the invention induce the generation or proliferation of glutathione and/or a member of the glutathione family, including, without limitation, glutathione peroxidase, and/or catalase synthesis.

In some embodiments of the invention, the glutathione modulators comprise, without limitation, schisandra chinensis berry, damiana and epimedium, maca, nettle leaf, Fe and Cu, and B-vitamins selected from the group comprising B₂, B₅, B₆ and B₇.

In a preferred embodiment of the invention, the neurotransmitter modulators of the invention induce and/or modulate the generation of neurotransmitters and modulates the transmission thereof by and between neurons and, hence, cells.

In some embodiments of the invention, the neurotransmitter modulators comprise, without limitation, nettle leaf, maca, eleuthero root, Yohimbe, cannabidiol (CBD), epimedium, and vitamins B₁ and B₆.

In a preferred embodiment of the invention, the DNA modulators of the invention support and/or enhance mitochondrial DNA activity.

In a preferred embodiment, the DNA modulators support and/or enhance mitochondrial DNA activity by protecting and/or facilitating the repair of mitochondrial DNA.

In a preferred embodiment of the invention, the DNA modulators comprise, without limitation, vitamin B₁₂.

In a preferred embodiment of the invention, the endocannabinoid system modulators induce cell receptor activity.

In a preferred embodiment, the endocannabinoid system modulators induce cannabinoid receptor activity.

In a preferred embodiment, the endocannabinoid system modulators comprise cannabidiol (CBD) or a component thereof.

In a preferred embodiment of the invention, the vibrational energy platforms of the invention comprise at least one energy signature component comprising or derived from, without limitation, schisandra chinensis, damiana leaf, eleuthero root, stinging nettle leaf, maca root, yohimbe root, epimedium, L-arginine, and L-citrulline.

The selection, function and synergistic relationship by and between the biochemical scaffold platforms and modulators associated therewith will now be described in detail.

### Vibrational Energy Platform

As indicated above, in a preferred embodiment of the invention, the vibrational energy platforms of the invention comprise at least one energy signature component derived from a selective herb, e.g., schisandra chinensis, or biological agent, e.g., L-arginine.

As set forth in Co-pending U.S. application Ser. No. 14/223,392, cellular transformation is measured by mathematical computations of the increased percentages (if any) of both pre- and post-oxygen and ATP levels with respect to each layered vibrational energy platform (VEP) application.

By way of example, for a diatomic molecule, the vibrational element can be approximated by the quantum harmonic oscillator, where the vibrational energy Ev is determined as follows:

Ev=(v+½)hv0,
where
v is an integer representing vibrational quantum numbers such that v=0, 1, 2, 3, . . . , where v=0 for a diatomic molecule at the ground vibrational state;
h is Planck's constant; and
v0 is the natural frequency of the harmonic oscillator.

Further, a diatomic molecule can be represented by the difference between the energy of the molecule idealized by setting the rotational energy equal to zero, and that of a further idealized molecule which is obtained by gradually stopping the vibration of the nuclei without placing any new constraint on the motions of electrons.

Another way a diatomic molecule can move is to have each atom oscillate or vibrate along a line (the bond) connecting the two atoms. The vibrational energy is approximately that of a quantum harmonic oscillator.

According to the invention, the energy signature components of the invention are chosen for their synergistic and intrinsic values to be of catalytic benefit in the cellular respiration process to produce adequate oxygen in the cell, thereby being of catalytic benefit in the production of the necessary energy (ATP).

As indicated above, in a preferred embodiment of the invention, the energy signature components comprise or are derived from, without limitation, schisandra chinensis, damiana leaf, eleuthero root, stinging nettle leaf, maca root, yohimbe root, epimedium, L-arginine, and L-citrulline.

As also set forth in Co-pending U.S. application Ser. No. 14/223,392, the noted herbs are employed to form stable biologically targeted energetic blanks, which can be generated as follows:

A composition comprising at least one of the aforementioned energy signature components and at least one B-vitamin comprising B₁, B₂, B₃, B₅, B₆, B₇, B₉ and B₁₂ is initially prepared.

After the composition is prepared, the composition is oscillated for 3-48 hours at a frequency in the range of approximately 23 Hz-1000 GHz, more preferably 102 GHz-250 GHz to obtain a positive charge.

In some embodiments of the invention, after the composition is placed in an appropriate storage container, the composition is subjected to further oscillation for approximately 3 hours at a frequency ranging from approximately 23 Hz-1000 GHz.

### Bioenergetic Platform

As indicated above, in a preferred embodiment of the invention, the bioenergetic platforms of the invention comprise at least one Krebs cycle modulator, glutathione modulator, neurotransmitter modulator, DNA modulator or endocannabinoid system modulator.

In some embodiments of the invention, the bioenergetic platforms comprise a Krebs cycle modulator and/or glutathione modulator and/or neurotransmitter modulator and/or DNA modulator and/or endocannabinoid system modulator.

Each of the noted modulators are discussed in detail below.

### Krebs Cycle Modulators

As indicated above, according to the invention, the Kreb cycle modulators of the invention induce and/or modulate at least one Krebs cycle metabolic reaction, process and/or pathway, including, without limitation, Krebs cycle product inhibition and/or substrate availability.

As set forth in U.S. application Ser. No. 14/233,392, a seminal process associated with the Krebs cycle is the catabolism of carbohydrates, fats and proteins, which results in the production of a two carbon organic product, i.e. acetate in the form of acetyl-CoA. Acetyl-CoA and two equivalents of water (H₂O) are consumed during the Krebs cycle, producing two equivalents of carbon dioxide (CO₂) and one equivalent of HS-CoA.

In addition, one complete cycle of the Kreb cycle converts three equivalents of nicotinamide adenine dinucleotide (NAD⁺) into three equivalents of reduced NAD⁺ (NADH), one equivalent of ubiquinone (Q) into one equivalent of reduced ubiquinone (QH₂), and one equivalent each of guanosine diphosphate (GDP) and inorganic phosphate (Pᵢ) into one equivalent of guanosine triphosphate (GTP). The NADH and QH₂ generated during the Kreb cycle are in turn used by the oxidative phosphorylation pathway to generate energy-rich adenosine triphosphate (ATP).

A primary source of acetyl-CoA is carbohydrates, which are broken down by glycolysis to produce pyruvate. Pyruvate is decarboxylated by the enzyme pyruvate dehydrogenase to generate acetyl-CoA.

Regulation of the Krebs cycle is largely dependent upon product inhibition and substrate availability. For example, NADH, a product of all dehydrogenases in the cycle (with the exception of succinate dehydrogenase) inhibits pyruvate dehydrogenase, isocitrate dehydrogenase, α-ketoglutarate dehydrogenase, and citrate synthase. Acetyl-coA inhibits pyruvate dehydrogenase, while succinyl-CoA inhibits alpha-ketoglutarate dehydrogenase and citrate synthase.

As indicated above, the Krebs cycle modulators of the invention are capable of inducing and/or modulating at least one Krebs cycle metabolic reaction, process and/or pathway, including, without limitation, product inhibition and/or substrate availability.

According to the invention, the Krebs cycle modulators can comprise, without limitation, ashwaganda, eleuthero root, maca, an amino acid, e.g., L-arginine and L-citrulline, and vitamins B₂, B₁, B₃, B₅ and B₉.

In some embodiments, the Krebs cycle modulators of the invention modulate product and/or substrate availability. By way of example, in some embodiments, the Krebs cycle modulators comprise eleuthero root, which Applicant has found facilitates the formation of glucose 6 phosphate. As stated, glucose 6 phosphate eventually converts to pyruvate, which enters into the Krebs cycle as Acetyl-coA.

In some embodiments, the Krebs cycle modulators comprise eleuthero root, which also enhances the activity of succinate dehydrogenase, an enzyme that facilitates the formation of FAD to FADH₂. These processes aid in the generation of ATP.

In some embodiments, the Krebs cycle modulators comprise maca. According to the invention, maca works synergistically with eleuthero root by inducing co-factor proliferation, which supports activation of the Krebs cycle.

Maca also facilitates the production of super oxide dismutase, i.e. an important antioxidant. Intracellular super oxide dismutase converts a highly undesirable free radical known as superoxide to hydrogen peroxide and oxygen.

In some embodiments, the Krebs cycle modulators comprise ashwaganda, which facilitates the lowering of cortisol and balancing of thyroid hormones. Ashwaganda also reduces the breakdown of ATP.

In some embodiments of the invention, the Krebs cycle modulators comprise an amino acid comprising, without limitation, L-arginine and L-citrulline. Applicant has found that L-arginine and L-citrulline facilitate the production of nitrous oxide. Nitrous oxide induces vasodilation and, hence, enhanced blood flow. The enhanced blood flow results in an increase in delivered O₂ and, thereby, enhanced cellular energy.

In some embodiments of the invention, the Krebs cycle modulators comprise a B-vitamin selected from the group comprising, without limitation, B₁, B₂, B₃, B₅ and B₉.

B₁, i.e. thiamine, plays a central role in the generation of energy from carbohydrates. B₁ is involved in RNA and DNA production, as well as nerve function. B1's active form is a coenzyme called thiamine pyrophosphate (TPP), which converts pyruvate to acetyl Coenzyme A (CoA).

B₂, i.e. riboflavin, is involved in energy production for the electron transport chain and catabolism of fatty acids, i.e. beta oxidation.

B₃, i.e. niacin, is composed of two co-enzyme forms of niacin: nicotinamide adenine dinucleotide (NAD) and nicotinamide adenine dinucleotide phosphate (NADP). Both play an important role in energy transfer reactions in the metabolism of glucose, fat and alcohol.

NAD carries H₂ and associated electrons during metabolic reactions, including the pathway from the Krebs cycle to the electron transport chain. NADP is a key coenzyme in lipid and nucleic acid synthesis.

B₅, i.e. pantothenic acid, is also involved in the oxidation of fatty acids and carbohydrates. Coenzyme A, which can be synthesised from panothenic acid, is involved in the synthesis of amino acids, fatty acids, ketones, cholesterol, phospholipids, steroid hormones, neurotransmitters, such as acetylcholine, and antibodies.

B₉, i.e. folic acid, acts as a co-enzyme in the form of tetrahydrofolate (THF), which is involved in the transfer of single-carbon units in the metabolism of nucleic acids and amino acids. THF is involved in pyrimidine nucleotide synthesis, which is required for normal cell division. Folate also aids in erythropoiesis, i.e. the production of red blood cells.

### Glutathione Modulators

According to the invention, the glutathione modulators of the invention induce (i) the generation or proliferation of glutathione and/or the glutathione family, including, without limitation, glutathione peroxidase, and/or (ii) catalase synthesis.

As also set forth in Co-pending U.S. application Ser. No. 14/233,392, glutathione; specifically, glutathione peroxidase, is an important intracellular antioxidant that induces conversion of hydrogen peroxide to H₂O and O₂. Glutathione reduces disulfide bonds formed within cytoplasmic proteins to cysteines by serving as an electron donor. In the process, glutathione is converted to its oxidized form glutathione disulfide (GSSG), as known as L-(−)-glutathione.

After oxidation, glutathione is reduced back to glutathione reductase, using NADPH as an electron donor.

As indicated above, the glutathione modulators of the invention induce the generation or proliferation of glutathione and/or a member of the glutathione family, including, without limitation, glutathione peroxidase.

According to the invention, the glutathione modulators can comprise, without limitation, schisandra chinensis berry, damiana and epimedium, and vitamin B₂.

As indicated above, B₂, i.e. riboflavin, facilitates energy production for the electron transport chain and catabolism of fatty acids, i.e. beta oxidation.

As also indicated above, in some embodiments of the invention, the glutathione modulator is effective to induce the synthesis of catalase, another key antioxidant. In these embodiments, the glutathione modulator can comprise, without limitation, maca, nettles leaves, Fe and Cu, and B-vitamins selected from the group comprising B₂, B₅, B₆ and B₇.

B₆, i.e. pyridoxine, is stored in the body as pyridoxal 5′-phosphate (PLP), which is the co-enzyme form of vitamin B₆. Pyridoxine is also involved in the metabolism of amino acids and lipids; in the synthesis of neurotransmitters and hemoglobin, as well as in the production of nicotinic acid (vitamin B₃). Pyridoxine also plays an important role in gluconeogenesis.

B7, i.e. biotin, also plays a key role in the metabolism of lipids, proteins and carbohydrates. It is a critical co-enzyme of four carboxylases: acetyl CoA carboxylase, which is involved in the synthesis of fatty acids from acetate; propionyl CoA carboxylase, which is involved in gluconeogenesis; β-methylcrotonyl Coa carboxylase, which is involved in the metabolism of leucin; and pyruvate CoA carboxylase, which is involved in the metabolism of energy, amino acids and cholesterol.

### Neurotransmitter Modulators

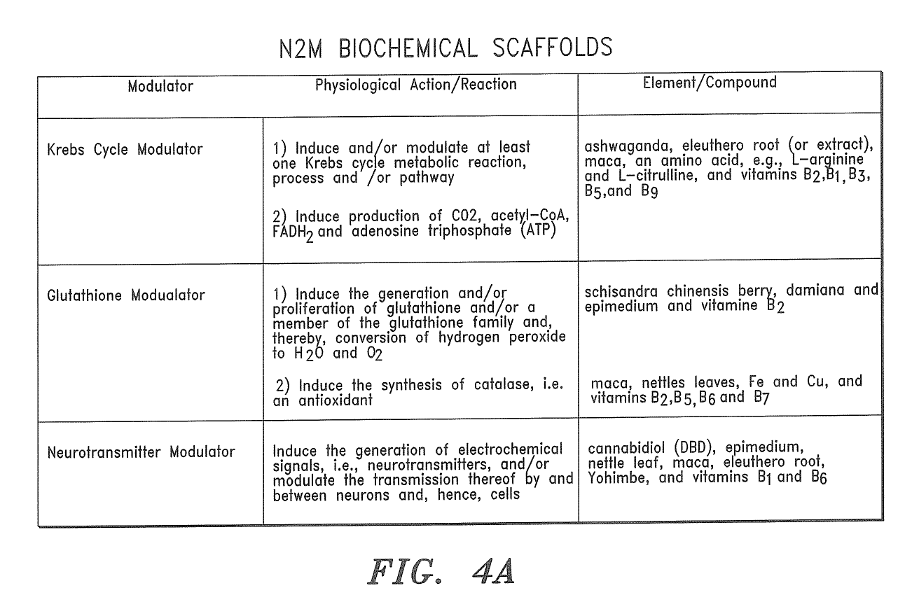

It is well established that the human brain contains large numbers of highly specialized cells called neurons. As illustrated in FIG. 3, the neurons 10 connect to and communicate with other neurons and, hence, cells via neurotransmitters 12, i.e. endogenous electrochemical signals, over synapses 14.

As further illustrated in FIG. 3 and discussed in detail below, when a sender neuron 10 generates and transmits neurotransmitters 12, the neurotransmitters 12 activate target receptors 16 on the receiver neuron 10 and, hence, cell.

According to the invention, the neurotransmitter modulators of the invention induce (and/or modulate) the generation and proliferation of neurotransmitters and modulate the transmission thereof by and between neurons and, hence, cells.

A key neurotransmitter is acetylcholine (ACh). Acetylcholine stimulates the central nervous system to enhance mental acuity, i.e. learning ability, short term memory and mental focus.

Another key neurotransmitter is dopamine. Dopamine functions as an inhibitory and excitatory neurotransmitter. As inhibitory neurotransmitter, it causes balance and general sense of well-being. As excitatory neurotransmitter, it improves cognition, concentration and focus.

A further key neurotransmitter is norepinephrine, which effects cognition, mood and mental concentration.

As indicated above, the neurotransmitter modulators of the invention induce the generation or proliferation of at least one neurotransmitter, including ACh, dopamine and norepinephrine, and/or the transmission thereof by and between neurons.

As also indicated above, the neurotransmitter modulators of the invention can comprise, without limitation, nettle leaf, maca, eleuthero root, Yohimbe, epimedium, cannabidiol (CBD), and vitamins B₁ and B₆.

Applicant has found that nettle leaf increases the level of neurotransmitters available to act on the neuron receptors; particularly, dopamine and acetylcholine, thus improving several mental processes, e.g. learning and recollection abilities.

Maca supports acetyl cholinesterase and, thereby, similarly enhances the proliferation of acetylcholine.

In addition to the Krebs cycle functions discussed above, eleuthero root enhances neuron activities, e.g., short term memory.

Yohimbe is a pre- and post-synaptic, alpha-2 adrenergic blocker that enhances neurotransmitter release and, thereby, enhanced cognitive functioning.

Yohimbe also induces elevation of norepinephrine from the locus coeruleus, resulting in enhanced memory. It is also been found that Yohimbe can abate one or more symptoms associated with post-traumatic stress disorder (PTSD).

The synergistic effect by and between maca and eleuthero root also provides cellular balance and decreases the negative effects of stress.

Epimedium, which includes the active element icariin, lowers the amyloid precursor protein (APP) level and, hence, reduces amyloid beta peptide (AB). Tau protein is used in the brain as axonal microtubule stabilizers. However, when hyperphosphorylated via the glycogen synthase kinase, amyloid beta proteins are generated and Alzheimer issues arise This process is initiated in the locus coeruleus. Icariin abates hyperphosphorylation and, thus, reduces AB.

Icariin is also an acetylcholinesterase inhibitor. Thus, more acetylcholine is available for memory and cognitive functions.

As discussed in detail below, cannabidiol (CBD) activates CB1 and CB2 receptors of the endocannabinoid system. By activating the CB1 and CB2 receptors the neurochemical consequences of the beta-amyloid proteins are reduced, which reduces inflammatory activity.

### DNA Modulators

According to the invention, the DNA modulators of the invention support and/or enhance mitochondrial DNA activity by, among other activities, protecting and/or facilitating the repair of mitochondrial DNA.

As indicated, mammalian mitochondria are organelles that produce more than 90% of cellular ATP. In addition to supplying ATP, i.e. cellular energy, mitochondria are also involved in other cellular mechanisms, including cellular differentiation, apoptosis, as well as cell cycle modulation and cell growth.

When a cell has temporarily or reversibly stopped dividing or regenerating it is often deemed to have entered a quiescent or senescent state referred to as the G₀ phase.

Non-proliferative cells generally enter the senescent G₀ phase or state from the G₁ phase and may remain senescent for long periods of time, possibly indefinitely (as is often the case for neurons). This is very common for cells that are fully differentiated.

The maximum number of cell divisions that a cell can undergo, varies from cell type to cell type and organism. In fibroblasts, this number is about 50 divisions, after which cell division ceases.

However, some cells become senescent after fewer replication cycles as a result of DNA damage or degradation, e.g., DNA mutations, DNA oxidation and chromosome losses, which would make a cell's progeny nonviable. If the DNA damage cannot be easily repaired, the cells either prematurely age or self-destruct (i.e. apoptosis or programmed cell death).

The process of cellular senescence can also be triggered by several additional mechanisms, including telomere shortening (i.e. a form of DNA damage or degradation).

Due to DNA replication mechanisms and oxidative stress, telomeres become progressively shorter with each round of replication. As increasing numbers of cell division occur, the telomeres reach a critically short length, which present as double-stranded DNA breaks, resulting in telomere-initiated senescence.

Protecting and/or facilitating the repair of mitochondrial DNA, which can be achieved by virtue of the DNA modulators of the invention, is thus essential to achieve optimal cell function and, thereby, physiological functioning. Healthy mitochondrial DNA also provides a healthy enzymatic process, which is required for oxidative phosphorylation and, hence, continued energy production.

As indicated above, the DNA modulators of the invention support mitochondrial DNA by protecting and/or facilitating the repair of mitochondrial DNA.

In a preferred embodiment of the invention, the DNA modulators of the invention comprise vitamin B₁₂.

According to the invention, B₁₂ supports DNA activity; specifically, synthesis and, in some instances, inhibits megaloblastic anemia.

B₁₂ is also involved in the cellular metabolism of carbohydrates, proteins and lipids. It functions as a co-enzyme in intermediary metabolism for the methionine synthase reaction with methylcobalamin, and the methylmalonyl CoA mutase reaction with adenosylcobalamin.

### Endocannabinoid System Modulators

According to the invention, the endocannabinoid system modulators of the invention induce cell receptor activity; preferably, cannabinoid receptor activity, i.e. receptors CB1 or CB2.

In a preferred embodiment of the invention, the endocannabinoid system modulators comprise cannabidiol (CBD).

CBD is one of many cannabinoid molecules produced by Cannabis, second only to THC in abundance.

CBD activates the two seminal cannabinoid receptors (CB1 and CB2) and, hence, as discussed below, induces several significant physiological activities. One significant physiological activity induced by activating the CB1 and CB2 receptors is modulation of inflammatory activity and diseases associated therewith, e.g. arthritis. The inflammation modulation, i.e. reduction thereof, is achieved by (among other factors) reducing the neurochemical effects of beta-amyloid proteins and, thereby, reactive oxidative stress and reactive oxygen.

As discussed below, in addition to activating the CB1 and CB2 receptors, CBD can, and in many instances will, enhance the levels of naturally-produced endocannabinoids, e.g., anandamide and 2-arachidonoyl glycerol (2-AG), by inhibiting the enzymes that break them down.

CBD also activates multiple serotonin receptors in the brain; particularly, serotonin 1A receptors. As a result, CBD can ameliorate various disorders, including neuropathic pain and motivational disorders, such as depression and anxiety.

CBD also modulates opioid receptor activity. As is well known in the art, opioid receptors are the key targets of pharmaceutical pain killers and drugs of abuse, such as morphine, heroin, and fentanyl. CBD's ability to modulate opioid receptor activity and enhance the activation of serotonin 1A receptors dampens drug cravings and, hence, can, and in many instances will, abate drug dependence; particularly, opioid and heroin dependence.

In addition to an endocannabinoid system modulator, CBD is also an effective neurotransmitter modulator. As indicated above. CBD activates the two seminal cannabinoid receptors CB1 and CB2. By activating the CB1 receptors, anandamide is increased and the associated elevation of corticosterone (stress hormone) and 2-arachidonoyl glycerol (2-AG) are reduced, which have a direct effect (and in many instances a calming effect) on the amygdala, i.e. emotional center.

Although CBD is a cannabinoid, CBD does not directly interact with and, hence, activate the CB1 and CB2 receptors. Instead, CBD indirectly activates the CB1 and CB2 receptors by modulating signaling through the CB1 and CB2 receptors by inhibiting the enzyme fatty acid amide hydrolase (FAAH). FAAH inactivates anandamide and also converts 2-AG to mono acylglycerol. By inhibiting FAAH more of anandamide and 2-AG available, which further enhances the calming effect on the amygdala.

The biochemical scaffolds of the invention thus modulate various seminal molecular activities, including inducing (i) at least one Krebs cycle metabolic reaction, process and/or pathway, (ii) generation or proliferation of glutathione and/or a member of the glutathione family, (iii) generation or proliferation of at least one neurotransmitter, and/or modulating the transmission thereof by and between neurons, (iv) inducing and/or supporting mitochondrial DNA activity and (v) cell receptor activity.

As also indicated above, by virtue of the noted modulated molecular activities that are induced by the biochemical scaffolds of the invention, cellular activity and function and, thereby, physical and mental function, is significantly enhanced. Indeed, it has also been found that administration of the biochemical scaffolds of the invention to a subject can, and in many instances will ameliorate various physical disorders, including neuropathic pain, inflammation and core temperature spikes, and mental disorders, such as post-traumatic stress disorder (PTSD), depression and anxiety. The biochemical scaffolds of the invention can also be employed to abate drug dependence; particularly, opioid and heroin dependence.

In one preferred embodiment of the invention, the biochemical scaffold is processed as set forth in U.S. application Ser. No. 14/233,392 or described herein, and preferably comprises the following bioenergetic platform, i.e. vitamins and herbs:

| Vitamin/Herbs | Milligrams | % of an Oz. |
|---|---|---|
| B₁₂ | 0.2-0.5 | ≤2.0%; preferably, approx. 0.7-1.7% |
| yohimbe | 650-1950 | ≤7.0%; preferably, approx. 2.3-6.9% |
| epimedium | 650-1950 | ≤7.0%; preferably, approx. 2.3-6.9% |
| ashwaganda | 650-1950 | ≤7.0%; preferably, approx. 2.3-6.9% |
| CBD | 5-20 | ≤1.0%; preferably, approx. 0.8-3.3% |

In some embodiments, the biochemical scaffold further comprises a cofactor, including, without limitation, organic cofactors, such as flavin and heme, and inorganic cofactor, such as the metal ions Mg²⁺, Cu⁺, Mn²⁺, and iron-sulfur clusters.

As indicated above, administration of the biochemical scaffolds of the invention to a subject will enhance cognitive function and/or ameliorate a physical or mental disorder.

Thus, in some embodiments of the invention there is provided a method of enhancing cognitive function comprising providing a biochemical scaffold of the invention and administering same to a subject.

In some embodiments of the invention there is provided a method treating a physical or mental disorder comprising providing a biochemical scaffold of the invention and administering same to a subject. According to the invention, the physical disorder can comprise, without limitation, neuropathic pain, inflammation, and core temperature spikes. The mental disorder can comprise, without limitation, post-traumatic stress disorder (PTSD), depression and anxiety.

In one preferred embodiment of the invention, there is thus provided a method of enhancing cognitive function of a subject comprising (i) providing a biochemical scaffold comprising a bioenergetic platform component and a vibrational energy platform component, the bioenergetic platform component comprising vitamin B₁₂, ashwaganda, yohimbe, epimedium and/or cannabidiol (CBD), the vibrational energy platform component comprising an energy signature component comprising schisandra chinensis, damiana leaf, eleuthero root, stinging nettle leaf, maca root, yohimbe, epimedium, L-arginine and/or L-citrullinen, the vibration energy component being subjected to harmonic oscillation at a frequency in the range of approximately 23 Hz-1000 GHz for a period of time in the range of approximately 3-48 hrs., and (ii) delivering the biochemical scaffold to the subject.

In some embodiments of the invention, the vibrational energy platform further comprises an energy signature component comprising B₁, B₂, B₃, B₅, B₆, B₇, B₉ or B₁₂.

In another embodiment of the invention, there is provided a method of treating PTSD of a subject that similarly comprises (i) providing a biochemical scaffold comprising a bioenergetic platform component and a vibrational energy platform component, the bioenergetic platform component comprising vitamin B₁₂, ashwaganda, yohimbe, epimedium and/or cannabidiol (CBD), the vibrational energy platform component comprising an energy signature component comprising schisandra chinensis, damiana leaf, eleuthero root, stinging nettle leaf, maca root, yohimbe, epimedium, L-arginine and/or L-citrullinen, the vibration energy component being subjected to harmonic oscillation at a frequency in the range of approximately 23 Hz-1000 GHz for a period of time in the range of approximately 3-48 hrs., and (ii) delivering the biochemical scaffold to the subject.

In some embodiments of the invention, the vibrational energy platform similarly further comprises an energy signature component comprising B₁, B₂, B₃, B₅, B₆, B₇, B₉ or B₁₂.

According to the invention, the biochemical scaffolds of the invention can be delivered to host tissue by various conventional means, including, without limitation, oral, sublingual, nasal, direct injection, topical application, etc.

In a preferred embodiment, the biochemical scaffolds are in liquid form.

In a preferred embodiment, a dose of the liquid form biochemical scaffold comprises in the range of approximately range of 0.006-0.070 oz, more preferable, in the range of approximately 0.006-0.018 oz.

As will readily be appreciated by one having ordinary skill in the art, the present invention provides numerous advantages compared to prior art formulations and methods for enhancing cell function and, thereby physiological performance. Among the advantages are the following:
The provision of biochemical scaffolds that enhance cell activity and function and, thereby, physical and mental function, by inducing at least one Krebs cycle metabolic reaction, process and/or pathway.
The provision of biochemical scaffolds that enhance cell activity and function and, thereby, physical and mental function, by inducing production of CO₂, acetyl-CoA, FADH₂ and adenosine triphosphate (ATP).
The provision of biochemical scaffolds that enhance cell activity and function and, thereby, physical and mental function, by inducing the generation of neurotransmitters and/or modulating the transmission thereof by and between neurons.
The provision of biochemical scaffolds that enhance cell activity and function and, thereby, physical and mental function, by inducing cell receptor activity.
The provision of biochemical scaffolds that enhance cell activity and function and, thereby, physical and mental function, by inducing cell receptor activity.
The provision of biochemical scaffolds that enhance cell activity and function and, thereby, physical and mental function, by modulating the endocannabinoid system.
The provision of biochemical scaffolds that enhance cell activity and function and, thereby, physical and mental function, by inducing and/or modulating mitochondria DNA activity.
The provision of biochemical scaffolds that induce generation and/or proliferation of glutathione and/or a member of the glutathione family and, thereby, conversion of hydrogen peroxide to H₂O and O₂.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A biochemical scaffold for modulating mammalian cell function, comprising:
a bioenergetic platform and a vibrational energy platform, said bioenergetic platform comprising a bioenergetic platform component selected from the group consisting of vitamin B₁₂, ashwaganda, yohimbe, epimedium and cannabidiol (CBD), said vibrational energy platform comprising an energy signature component selected from the group consisting of schisandra chinensis, eleuthero root, maca root, L-arginine and L-citrulline.

2. The biochemical scaffold of claim 1, wherein said energy signature component further comprises a B-vitamin selected from the group consisting of B₁, B₂, B₃, B₅, B₆, B₇, B₉ and B₁₂.

3. The biochemical scaffold of claim 1, wherein said energy signature component is subjected to harmonic oscillation at a frequency in the range of approximately 23 Hz-1000 GHz for a period of time in the range of approximately 3-48 hrs.

4. A method of treating post-traumatic stress disorder (PTSD) of a subject, comprising the steps of:
providing a biochemical scaffold comprising a bioenergetic platform and a vibrational platform, said bioenergetic platform comprising a bioenergetic platform component selected from the group consisting of vitamin B₁₂, ashwaganda, yohimbe, epimedium and cannabidiol (CBD), said vibrational energy platform comprising an energy signature component selected from the group consisting of schisandra chinensis, eleuthero root, maca root, L-arginine and L-citrulline; and
delivering said biochemical scaffold to the subject.

5. The method of claim 4, wherein said energy signature component further comprises a B-vitamin selected from the group consisting of B₁, B₂, B₃, B₅, B₆, B₇, B₉ and B₁₂.

6. The method of claim 4, wherein said energy signature component is subjected to harmonic oscillation at a frequency in the range of approximately 23 Hz-1000 GHz for a period of time in the range of approximately 3-48 hrs.
